(19) United States
(12) Patent Application Publication
(10) Pub. No.: US 2006/0008808 A1
(43) Pub. Date: Jan. 12, 2006

(54) Compositions and methods for enhancing an immune response

(76) Inventors: De Yang, Frederick, MD (US); Joost J. Oppenheim, Bethesda, MD (US); Helene F. Rosenberg, Bethesda, MD (US); O.M. Zack Howard, Frederick, MD (US)

Correspondence Address: LEYDIG, VOIT & MAYER, LTD., TWO PRUDENTIAL PLAZA, SUITE 4900, 180 NORTH STETSON AVENUE, CHICAGO, IL, 60601-6780, US

(21) Appl. No.: 10/834,733

(22) Filed: Apr. 29, 2004

Related U.S. Application Data
(60) Provisional application No. 60/466,796, filed on Apr. 29, 2003.
(60) Provisional application No. 60/466,797, filed on Apr. 29, 2003.

(51) Int. Cl. C12Q 1/68

(52) U.S. Cl. 435/6

(57) ABSTRACT

The invention provides compositions and methods of enhancing an immune response in a subject. The compositions comprise eosinophil derived-neurotoxin (EDN) and/or human pancreatic ribonuclease (hPR), which can be used as adjuvants to elicit an enhanced immune response in a subject.

### CROSS-REFERENCE TO RELATED PATENT APPLICATIONS

This patent application is a continuation-in-part of copending U.S. patent application Ser. No. 10/396,317, filed Mar. 26, 2003, which claims the benefit of U.S. Provisional Patent Application No. 60/393,110, filed Jul. 3, 2002, and U.S. Provisional Patent Application No. 60/394,511, filed Jul. 10, 2002. This patent application claims the benefit of U.S. Provisional Patent Application No. 60/466,796, filed Apr. 29, 2003, and U.S. Provisional Patent Application No. 60/466,797, filed Apr. 29, 2003.

### FIELD OF THE INVENTION

This invention pertains to compositions and methods for enhancing an immune response in a subject.

### BACKGROUND OF THE INVENTION

For the induction of an immune response to any given antigen, dendritic cells have to be mobilized to the site of antigen entry where they engulf, process, and display antigenic epitopes on their surface together with MHC class II molecules. Subsequently, dendritic cells bearing antigenic epitopes travel to secondary lymphoid organs (such as spleen and lymph nodes), to stimulate, and to sustain the activation of naïve T lymphocytes. Most, if not all, soluble antigens, such as tumor-associated antigens or purified microbial antigens designed for vaccination, do not themselves have the capacity to mobilize or to mature dendritic cells, and therefore do not induce a successful protective immune response if administered alone. Potent immune responses to these antigens can be induced if given simultaneously with immunoadjuvants, which are reagents that can enhance the immunogenicity of co-administered antigens. In animal experiments, the most often used adjuvants include Freund's complete adjuvant, incomplete Freund's adjuvant, and pertussis toxin. However, these adjuvants cannot be used in humans due to their toxicity. Alum is the only adjuvant being used in humans, but its adjuvanticity is rather weak.

Therefore, there remains a need for an adjuvant that can be used to enhance an immune response in a subject. The present invention provides such an adjuvant. These and other advantages of the invention, as well as additional inventive features, will be apparent from the description of the invention provided herein.

### BRIEF SUMMARY OF THE INVENTION

The invention provides a composition comprising an immunogenic antigen and eosinophil-derived neurotoxin (EDN) or human pancreatic ribonuclease (hPR).

The invention also provides a method of enhancing an immune response in a subject, comprising administering to the subject a pharmaceutical composition comprising a therapeutically effective amount of EDN or hPR and a pharmaceutically acceptable carrier, whereby the administration of the composition enhances the immune response in the subject.

Further provided by the invention is a method of enhancing an immune response to a vaccine in a subject, comprising administering to the subject (a) a pharmaceutical composition comprising a therapeutically effective amount of EDN or hPR and a pharmaceutically acceptable carrier and (b) a vaccine, whereby the administration of the composition enhances the immune response to the vaccine in the subject as compared to the immune response in the subject that results from administration of the vaccine without the composition.

Additionally, the invention provides a method for inducing dendritic cell (DC) chemotaxis in a subject that would benefit from such induction, wherein the method comprises administering to the subject a pharmaceutical composition comprising a therapeutically effective amount of EDN and a pharmaceutically acceptable carrier, such that DC chemotaxis is induced in the subject.

The invention also provides a method for activating DCs, monocytic cells, or a combination thereof, wherein the method comprises administering to the subject a pharmaceutical composition comprising a therapeutically effective amount of EDN or hPR and a pharmaceutically acceptable carrier, such that DCs, monocytic cells, or a combination thereof are activated in the subject.

Moreover, the invention provides a method for inducing maturation of DCs in a subject, wherein the method comprises administering to the subject a pharmaceutical composition comprising a therapeutically effective amount of EDN or hPR and a pharmaceutically acceptable carrier, such that maturation of DCs is induced in the subject.

### BRIEF DESCRIPTION OF THE DRAWINGS

FIG. 1 illustrates eosinophil-derived neurotoxin (EDN) induction of human immature dendritic cells (iDC) migration. FIG. 1A illustrates the dose dependent migration of iDCs in response to EDN. Chemotaxis medium (CM) alone (control), 10, 100, or 1000 ng/mL of EDN, or 100 ng/mL of stromal cell-derived factor (SDF)-1α were added to the lower wells of the chemotaxis chambers. FIG. 1B illustrates the inhibition of EDN-induced iDC migration by pertussis-toxin (PTX), wherein iDCs were preincubated in the absence (PTX−) or presence (PTX+) of 200 ng/mL of PTX prior to chemotaxis assay. DC migration is shown as the average migrated cells (mean±standard deviation) of triplicate wells. Chemotaxis of iDCs is shown as the average cell migration (mean±standard deviation) of triplicate wells.

FIG. 2 illustrates the chemotaxis of human DCs in response to EDN and mouse eosinophil-associated RNase 2 (mEAR2). FIG. 2A illustrates the results when 0, 10, 100, or 1000 units/mL of RNase inhibitor (RI) was mixed with 1000 ng/mL of EDN or 100 ng/mL of SDF-1α, and the mixtures were added to the lower wells of chemotaxis chambers. FIG. 2B illustrates the chemotactic activities of several members of the RNase A superfamily (EDN, human pancreatic ribonuclease (hPR), human angiogenin (hANG), mEAR2, and bovine pancreatic ribonuclease (bPR)) at concentrations ranging from 0 to 5000 ng/mL. Chemotaxis of iDCs is shown as the average cell migration (mean±standard deviation) of triplicate wells.

FIG. 3 illustrates the target cell spectrum for EDN. FIG. 3A illustrates the migration of CD34+ cell derived DC precursors (preDCs), iDCs, or mature DCs (mDCs) in response to EDN evaluated by chemotaxis assay. FIG. 3B illustrates the migration of monocytes (Mo), monocyte-derived immature dendritic cells (Mo-iDCs), and monocyte-derived mature dendritic cells (Mo-mDCs) in response to EDN evaluated by chemotaxis assay. Cell migration is shown as the average (mean±standard deviation) number of cells migrated in triplicate wells. The error bars are not evident if they are smaller than the size of the symbols.

FIG. 4 illustrates that EDN and mEAR2 function as chemoattractants for mouse DCs. The migration of iDCs and mDCs generated from mouse bone marrow hematopoietic progenitor cells (HPCs) in response to EDN or mEAR2 was investigated by chemotaxis assay and the results are shown as the average cell migration (mean±standard deviation) of triplicate wells. FIG. 4A illustrates the response of cell migration (iDCs or mDCs) to 0, 1, 10, 100, or 1000 ng/mL of EDN or mEAR2. FIG. 4B illustrates the blockade of EDN- or mEAR2-induced migration of iDCs by the presence of placental RI at 1000 U/mL. FIG. 4C illustrates the inhibition of EDN-induced mDC migration by PTX, wherein mDCs were preincubated in the absence (PTX−) or presence (PTX+) of 200 ng/mL of PTX prior to chemotaxis assay. In FIGS. 4B and 4C, 1000 ng/mL of EDN or mEAR2 was used.

FIG. 5 illustrates the alignment of the amino terminal sequences of human EDN (hEDN), mEAR2, human eosinophil cationic protein (hECP), hPR and hANG. Regions of identical sequence shared by EDN and mEAR2 are identified with open boxes, and if shared by ECP, hPR, and HANG, the boxes are extended to include them. The additional amino-terminal residues of the “−4” form of EDN are included in parentheses. An arrow indicates the position of the universally conserved histidine that serves as a crucial catalytic residue in all RNase A superfamily ribonucleases.

FIG. 6 illustrates the in vivo chemotactic activity of mEAR2. The average of total infiltrating cells or CD11c and I-A/I-E double-positive DCs per pouch are presented for mice injected with (i) phosphate buffered saline (PBS) alone, (ii) hANG and PBS, or (iii) mEAR2 and PBS.

FIG. 7 illustrates chemokine production from CD34+ progenitor-derived iDCs induced by EDN (•) and hPR (▴). FIG. 7A illustrates the time course analysis for 10 selected chemokines. Immature DCs (10⁶/mL) were cultured in G4 medium containing EDN or hPR (1 μg/mL) for various time periods, ranging from 0 to 48 hours, and the induction of the selected chemokines is presented as Cy5 intensity (log). FIG. 7B illustrates the dose response analysis for the 10 selected chemokines. Immature DCs (10⁶/mL) were cultured in G4 medium for 48 hours in the absence or presence of various concentrations of EDN or hPR, ranging from 0 to 1000 ng/mL, and the induction of the selected chemokines is presented as Cy5 intensity (log).

FIG. 8 illustrates cytokine production from CD34+ progenitor-derived iDCs induced by EDN (•) and hPR (▴). FIG. 8A illustrates the time course analysis for 5 selected cytokines. Immature DCs (10⁶/mL) were cultured in G4 medium containing EDN or hPR (at 1 μg/mL) for various time periods, ranging from 0 to 48 hours, and the induction of the selected cytokines is presented as Cy5 intensity (log). FIG. 8B illustrates the dose response analysis for the 5 selected cytokines. Immature DCs (10⁶/mL) were cultured in G4 medium for 48 hours in the absence or presence of various concentrations of EDN or hPR, ranging from 0 to 1000 ng/mL, and the induction of the selected cytokines is presented as Cy5 intensity (log).

FIG. 9 illustrates the induction of DC maturation by hPR and EDN. FIG. 9A illustrates the surface expression of CD14, CD40, CD83, CD86, and HLA-DR by monocyte-derived DCs cultured for 48 hours with (a) G4 alone, (b) G4 and hPR (at 1 μg/mL), or (c) G4 and EDN (at 1 μg/mL). FIG. 9B illustrates the differences in chemotactic responses of monocyte-derived DCs cultured for 48 hours in the absence or presence of 1 μg/mL of EDN or hPR to optimal concentrations (100 ng/mL) of RANTES or Secondary Lymphoid-Tissue Chemokine (SLC).

### DETAILED DESCRIPTION OF THE INVENTION

Eosinophil-derived neurotoxin (EDN) is a protein belonging to the ribonuclease (RNase) A superfamily, which has recently been found to have antiviral activity against respiratory syncytial virus and human immunodeficiency virus in vitro. Many molecules belonging to the RNase superfamily have been identified, which, based on their evolutionary relationship, have been classified into five subfamilies: (1) eosinophil-associated ribonucleases (EARs), which include human EDN and human eosinophil cationic protein (ECP), (2) pancreatic-type ribonucleases (PRs), (3) ribonuclease 4s (R4s), (4) angiogenins (ANGs), and (5) ribonucleases from the bullfrog Rana (RRs), which include onconase.

Many cell types, such as eosinophils, neutrophils, and peripheral blood mononuclear cells, produce EDN, which is therefore likely to be present at sites of viral infection and inflammation. Based on the results of experiments to evaluate the effect of EDN on the migration of human leukocytes, EDN induces chemotaxis of dendritic cells (DCs) (e.g., CD34+ progenitor-derived or monocyte-derived DCs). EDN-induced DC chemotaxis is inhibited by either pretreatment of DCs with pertussis toxin (PTT), a selective Giα protein inhibitor, or placental RNAse inhibitor. Thus, EDN induces chemotaxis of DCs using a Giα protein-coupled receptor. The capacity of DCs to respond chemotactically to EDN is maintained even after maturation. However, leukocytes other than DCs (including neutrophils, monocytes, DC precursors, and CD3+ T cells) do not respond to EDN in this fashion. EDN also induces the activation of MAPK in human DCs, a characteristic shared by many chemotactic factors.

Although EDN can be expressed by liver, spleen, neutrophils, as well as peripheral blood mononuclear cells, EDN is predominantly released or produced upon eosinophil degranulation or activation. Since heavy infiltration and activation of eosinophils are most often seen in tissues with helminth or viral infections or allergic reactions, EDN is potentially produced at considerable levels at sites of inflammation resulting from helminth or viral infection and allergic reactions. Based on its multifunctional activities, EDN can contribute to both innate and adaptive immunity in the host. Since EDN is chemotactic for iDCs, EDN can contribute to the recruitment of DCs to sites of helminth, virus, and allergen entry, thereby enhancing antigen-specific immunity by promoting antigen uptake, processing, and ultimately presentation. EDN also contributes to the maturation of DCs, and therefore can promote trafficking of DCs to regional lymph nodes and antigen presentation. Recruitment and activation of both iDCs and mDCs at sites of helminth and allergic antigen entry can promote the initiation of the immune response in situ, resulting in the formation of “tertiary lymphoid tissue,” such as granuloma formulation around helminth eggs. Additionally, EDN can contribute to the induction of proinflammatory mediators such as interleukin (IL)-1β, IL-6, tumor necrosis factor (TNF)-α, epithelial neutrophil activating peptide (ENA)-78, IP-10, monocyte chemoattractant protein (MCP)-2, MCP-3, macrophage inflammatory protein (MIP)-1α, MIP-1β, and Rantes to amplify local inflammatory responses.

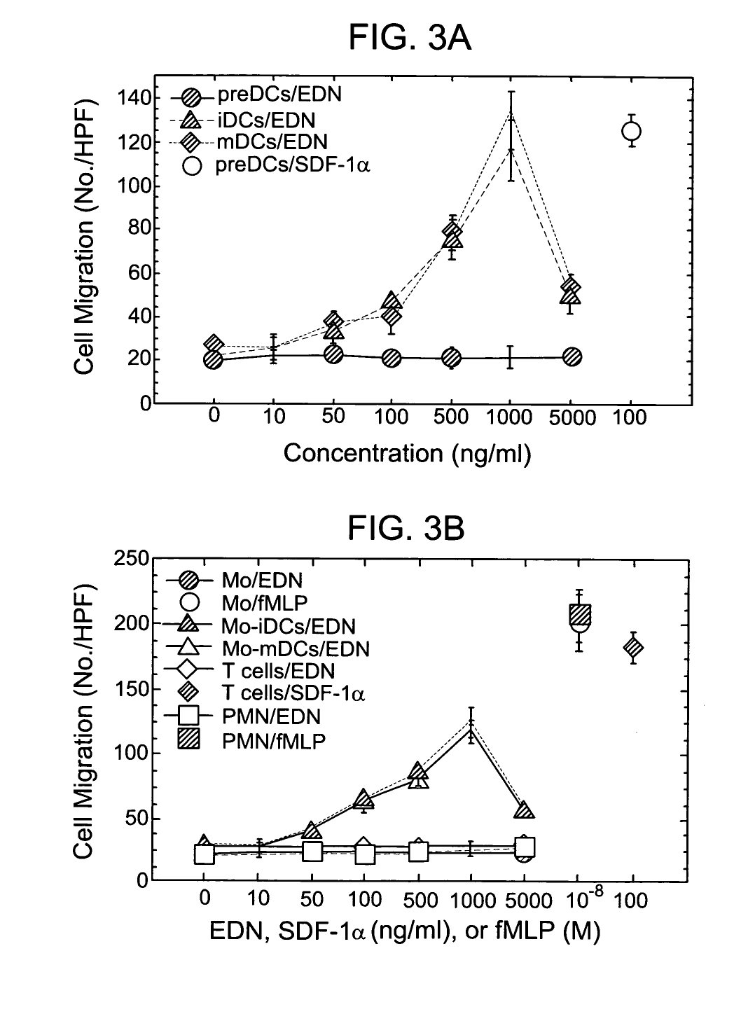

Screening a number of proteins belonging to the RNase A superfamily revealed that the mouse eosinophil-associated RNase (mEAR2), one of a cluster of divergent orthologs of human EDN, is also chemotactic for iDCs and mDCs. EDN and mEAR2 are unique chemoattractants for DCs, since neither one acts on neutrophils, monocytes, or T cells. Of all the chemokines and classical chemoattractants that are active on DCs, only four (i.e., stromal cell derived factor (SDF)-1α, platelet-activating factor, complement C5a, and formyl-methionine-leucine-phenylalanine (fMLP)) are chemotactic for both iDCs and mDCs. However, SDF-1α, platelet-activating factor, C5a, and fMLP are also chemotactic for diverse types of leukocytes. For example, SDF-1α is chemotactic for DC precursors (FIG. 3A) and lymphocytes. Platelet-activating factor, C5a, and fMLP are chemotactic for neutrophils and monocytes.

Another member of the RNase family, human pancreatic ribonuclease (hPR), like EDN, has the capacity to activate DCs (e.g., CD34+ progenitor-derived and monocyte-derived DCs) and monocytic cells, leading to the production of various mediators including cytokines, chemokines, growth factors, and soluble receptors. Human PR, like EDN, also induces the maturation of DCs (e.g., as evidenced by the upregulation of CD83, CD86, and HLA-DR, and functional chemokine receptor, CCR7). These similarities to EDN are surprising, since hPR does not display chemotactic or antiviral activity, like EDN.

Most of the mediators induced by EDN or hPR from human DCs and monocytic cells are proinflammatory cytokines and chemokines, such as ENA-78, IL-12, IL-6, IP-10, MCP-2, MCP-3, MIP-1α, MIP-1β, Rantes, TNF-α, B lymphocyte chemoattractant (BLC), I-309, IL-1β, MDC, TARC. Although similar sets of mediators are induced from DCs by EDN and hPR, there are some differences. For example, hPR does not induce ENA-78 or IL-12p70 production, and EDN, but not hPR, induces the production of monocyte induced by interferon γ (MIG) and TNF-α. Furthermore, EDN and hPR differentially regulate the mRNA levels of several cytokines, including IL-5, IL-8, IL-12p35, and TNF-α. Additionally, there are marked differences in the production of soluble mediators by the monocytic cell line, THP-1, after EDN- or hPR-stimulation.

Due to these effects on the immune system, EDN or hPR can be used as, or developed into, a clinically applicable adjuvant (immunoadjuvant) for the promotion of an immune response to any antigen of interest, such as those derived from tumors, viruses, or pathological microbial invaders. An “adjuvant” refers to a substance that non-specifically enhances the immune response to an antigen. For example, the DC chemoattracting activity of EDN is likely to promote the accumulation of DCs to sites of a co-administered antigen thereby enhancing antigen uptake and processing. Since EDN's chemoattracting activity is rather selective, other types of leukocytes are less likely to be attracted, which should help to limit unwanted side effects. EDN and hPR can enhance the migration of DCs bearing antigenic epitopes to secondary lymphoid organs and subsequent antigen presentation based on their capacity to induce the phenotypic maturation of DCs. Furthermore, EDN and hPR can enable the DCs to sustain the activation of naïve T lymphocytes by stimulating the production of cytokines and chemokines from DCs.

Accordingly, one embodiment of the invention is a composition (e.g., a pharmaceutical composition) comprising an immunogenic antigen and EDN, hPR, compositions thereof, or combinations thereof, which can be used, e.g., to vaccinate a subject.

Another embodiment of the invention is a method of enhancing an immune response in a subject, comprising administering a composition (e.g., a pharmaceutical composition) comprising a therapeutically effective amount of EDN or hPR to the subject, whereby the administration of the composition comprising EDN or hPR enhances the immune response in the subject.

EDN, hPR, or compositions thereof can be used as an adjuvant to enhance an immune response in a subject in several ways, including but not limited to: (1) as a composition (e.g., a pharmaceutical composition) with an immunogenic antigen, (2) as a fusion protein or chemical conjugation with an immunogenic antigen, and (3) as a EDN-expressing DNA construct or hPR-expressing DNA construct (either separately or fused with an immunogenic antigen) which can be applied directly (e.g., with a DNA delivery device such as a GeneGun). The immunogenic antigen can be any antigen of interest, such as an antigen derived from tumors, pathological microbial invaders, viruses, allergens, and the like.

Additionally, EDN, hPR, or active fragments, fusions, and derivatives thereof, can be administered to a subject (e.g., as a composition, such as a pharmaceutical composition) without an antigen to enhance an immune response in a subject. For example, to enhance an immune response against a tumor within a subject, EDN, hPR, or compositions thereof can be injected directly into the tumor or in an area surrounding the tumor, where tumor antigens are located.

“Active fragments, fusions, and derivatives thereof” refer to fragments, fusions, and derivatives of EDN and hPR that retain sufficient activity of one or more of EDN's or hPR's functions. For example, active fragments, fusions, and derivatives of EDN retain sufficient activity to (a) induce DC chemotaxis, (b) induce DC and monocyte activation, and (c) induce DC maturation. Active fragments, fusions, and derivatives of hPR retain sufficient activity to (a) induce DC and monocyte activation and (b) induce DC maturation.

The invention also provides a method of enhancing an immune response to a vaccine in a subject, comprising administering to the subject a composition (e.g., a pharmaceutical composition) comprising a therapeutically effective amount of EDN or hPR and the vaccine, whereby the administration of the composition enhances the immune response to the vaccine relative to the immune response to the vaccine when without the administration of the composition.

A “vaccine” refers to a preparation of antigen, often combined with adjuvants, that is administered to individuals to induce protective immunity against infections. The antigen may be in the form of live, but avirulent, microorganisms or viruses, killed microorganisms or denatured viruses, purified macromolecular components of a microorganism or virus, or a plasmid that contains a nucleic acid molecule encoding an antigen.

A “subject” refers to an animal (which includes a human), such as domesticated animals (e.g., cats, dogs, and the like), livestock (e.g., cattle, horses, pigs, sheep, goats, and the like), laboratory animals (e.g., mouse, rabbit, rat, guinea pig, and the like) and birds. Preferably, the subject is a mammal such as a primate, and more preferably, the subject is a human.

An “immune response” refers to the response by the immune system of a subject to an organism, protein, or other substance that is recognized as foreign to the subject. For example, an immune response may include, but is not limited to, the mobilization of leukocytes to a site of entry into the subject by a foreign body (e.g., a foreign protein, virus, or microbe), the release of antimicrobial substances by cells of the immune system, the release of chemotactic substances by cells of the immune system which attract other immune cells to the site of infection, and the release of chemokines by cells of the immune system, separately or in combination.

Those skilled in the art can detect an enhanced immune response in a subject. For example, a person of skill in the art can detect an increase in the number of DCs attracted to the site of inoculation, an increase in antibodies directed to a specific antigen that is administered with EDN, hPR, or compositions thereof, and an increase in the number of T cells directed against a specific antigen. Moreover, if a tumor antigen is injected into a subject with EDN, hPR, or compositions thereof, those skilled in the art can detect enhancement of the adaptive immune response by detecting a decrease in size or growth rate of a tumor.

The method of enhancing an immune response in a subject can be used to enhance an immune response against bacterial infections, viral infections, fungal infections, cancer, allergic reactions, and the like.

The composition comprising EDN or hPR (or active fragments, fusions, or derivatives thereof) is preferably a pharmaceutical composition, wherein the components of the composition are combined with a pharmaceutically acceptable carrier. The term “pharmaceutically acceptable” refers to a non-toxic material that does not interfere with the effectiveness of the biological activity of the active ingredient(s). The characteristics of the carrier to be added to the composition depend on the particular route of administration of the pharmaceutical composition to be employed. Such a composition additionally can contain diluents, fillers, salts, buffers, stabilizers, preservatives, antioxidants, solubilizers, and other materials known in the art. Additionally, the composition can contain other active ingredients, such as cytokines, lymphokines, or other hematopoietic factors (e.g., macrophage colony-stimulating factor (M-CSF), granulocyte/macrophage colony stimulating factor (GM-CSF), IL-1, IL-2, IL-3, IL-4, IL-5, IL-6, IL-7, IL-8, IL-9, IL-10, IL-12, IL-13, IL-14, IL-15, granulocyte colony-stimulating factor (G-CSF), megakaryocyte colony stimulating factor (Meg-CSF), stem cell factor, and erythropoietin. Such additional factors and/or agents can be included in the pharmaceutical composition to produce a synergistic effect with EDN or hPR, or to minimize potential side effects.

The pharmaceutical composition of the invention can be in the form of a liposome in which the active ingredient(s) is combined with, in addition to other pharmaceutically acceptable carriers, amphipathic agents, such as lipids, which exist in aggregated form as micelles, insoluble monolayers, liquid crystals, or lamellar layers in aqueous solution. Suitable lipids for liposomal formulation include, but are not limited to, monoglycerides, diglycerides, sulfatides, lysolecithin, phospholipids, saponin, bile acids, lipid extracted from whole cells, and the like. Preparation of such liposomal formulations is known in the art, as disclosed in, e.g., U.S. Pat. No. 4,235,871, U.S. Pat. No. 4,501,728, U.S. Pat. No. 4,837,028, and U.S. Pat. No. 4,737,323.

EDN, hPR, or compositions thereof can be administered (e.g., with a pharmaceutically acceptable carrier) in doses, for example, from about 0.01 μg to about 100 mg per day (e.g., about 0.05 μg, about 0.1 μg, about 0.5 μg, about 1 μg, about 5 μg, about 10 μg, about 50 μg, about 100 μg, about 500 μg, about 1 mg, about 2 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or ranges therebetween), depending on the sex and size of the subject, and nature and severity of the underlying disease or condition to be treated, and the nature of the prior treatment which the subject has undergone. The attending physician of the subject can decide the amount of EDN, hPR, or compositions thereof with which to treat each individual subject. Initially, the attending physician will administer low doses of EDN, hPR, or compositions thereof, and observe the subject's response. Larger doses of EDN, hPR, or compositions thereof can be administered until the optimal therapeutic effect is obtained for the subject, and at that point, the dosage is not increased further. Dosage schedules are at the discretion of the attending physician, and can vary depending on the dosage form, dosage amount, route of administration, severity of the condition, and other factors. Dosage schedules can vary from about every 4 hours to about once per day (e.g., about every 6 hours, about every 8 hours, about every 12 hours, about every 16 hours, about every 20 hours).

The composition (e.g. pharmaceutical composition) comprising EDN or hPR preferably contains a therapeutically effective amount of EDN or hPR. “Therapeutically effective amount” refers to the total amount of each active component of the pharmaceutical composition that is sufficient to demonstrate meaningful benefit in a subject. When applied to an individual active ingredient that is administered alone, the term refers to that ingredient alone. When applied to a combination, the term refers to the combined amounts of the active ingredients that result in the therapeutic effect, whether administered in combination, serially, or simultaneously.

Routes of administration of EDN, hPR, and compositions thereof can be in vivo, in vitro, or ex vivo as known in the art. Preferred routes of administration include, but are not limited to, oral administration, inhalation, cutaneous administration, and parenteral administration, including subcutaneous, intracutaneous, intramuscular, intra-organ, and intravenous injection.

When a therapeutically effective amount of EDN, hPR, or compositions thereof is administered orally, the EDN, hPR, or compositions thereof can be in the form of a tablet, capsule, powder, solution, or elixir. When administered in the tablet form, the pharmaceutical composition can additionally contain a solid carrier, such as gelatin or an adjuvant. The tablet, capsule, and powder can contain from about 5 to about 95% (e.g., about 10%, about 20%, about 25%, about 30%, about 35%, about 40%, about 45%, about 50%, about 55%, about 60%, about 65%, about 70%, about 75%, about 80%, about 85%, about 90%, or ranges therebetween) of EDN or hPR, on a weight basis.

When a therapeutically effective amount of EDN, hPR, or compositions thereof is administered in liquid form, a liquid carrier such as water, petroleum, oils of animal or plant origin (e.g., peanut oil, mineral oil, soybean oil, sesame oil, or synthetic oils) can be added. The liquid form of the pharmaceutical composition can also contain physiological saline solution, dextrose, or other saccharide solution, and/or glycols, such as ethylene glycol, propylene glycol, or polyethylene glycol. When administered in liquid form, the pharmaceutical composition contains from about 0.5 to about 90% (e.g., about 1%, about 5%, about 10%, about 20%, about 25%, about 30%, about 35%, about 40%, about 45%, about 50%, about 55%, about 60%, about 65%, about 70%, about 75%, about 80%, about 85%, or ranges therebetween) of EDN or hPR, and preferably from about 1 to about 50% of EDN or hPR, on a weight basis.

When a therapeutically effective amount of EDN, hPR, or compositions thereof is administered by parenteral injection (e.g., intravenous, cutaneous, or subcutaneous injection), the EDN, hPR, or compositions thereof are in the form of a pyrogen-free, parenterally acceptable aqueous solution. The preparation of such parenterally acceptable solutions, with the proper pH, isotonicity, stability, and the like, is within the skill in the art. A preferred pharmaceutical composition for parenteral injection can contain, in addition to EDN or hPR, an isotonic vehicle such as Sodium Chloride Injection, Ringer's Injection, Dextrose Injection, Dextrose and Sodium Chloride Injection, Lactated Ringer's Injection, or other vehicles, many of which are known in the art.

The duration of therapy using the pharmaceutical composition of the invention will vary, depending on the severity of the disease or condition being treated and the idiosyncratic response of each individual patient. The attending physician will decide on the appropriate duration of therapy using the pharmaceutical composition of the invention.

The compositions of the invention may be administered in accordance with the method of the invention either alone or in combination with other therapies, such as treatments employing cytokines, lymphokines, or other hematopoietic factors. When co-administered with one or more cytokines, lymphokines or other hematopoietic factors, the chemotactic protein or peptide may be administered either simultaneously or sequentially with the cytokine(s), lymphokine(s), other hematopoietic factor(s), thrombolytic factor(s), or anti-thrombotic factor(s).

When EDN, hPR, or compositions thereof are used in combination with the administration of an antigen (e.g., in vaccination), the antigen can be delivered in accordance with any suitable method, many of which are known. EDN, hPR, or compositions thereof can be administered at the time of, before, or after the administration of the antigen. Simultaneous administration can be made in a single composition comprising antigen and EDN, hPR, or compositions thereof, or in separate preparations.

When the compositions of the invention are used to induce an immune response to an antigenic agent (e.g., a tumor antigen, infectious agent, or other diseased tissue) in the subject, the composition is preferably administered in a manner that attracts DCs and/or T cells to the site of the antigenic agent. For example, EDN or compositions thereof can be injected into or a tumor or the region of a tumor in order to promote migration of DCs and/or T cells to the tumor site. Similarly, EDN or compositions thereof can be administered in an area of infection by a virus or bacteria to promote migration of DCs and/or T cells to the site of infection.

The invention is also directed to a method for inducing DC chemotaxis in a subject that would benefit from such induction, wherein the method comprises administering to the subject a pharmaceutical composition comprising a therapeutically effective amount of EDN, such that DC chemotaxis is induced in the subject.

EDN, fragments, fusions, and derivatives thereof (collectively referred to as chemotactic agents) are useful for the treatment of conditions, which would benefit from stimulation or induction of DC chemotaxis, either to a site of desired cell action or away from a site of undesired cell action. For example, there are many situations in which it would be desirable to recruit or mobilize DCs and/or T cells to a particular site where their presence or action is desired. Such situations include, for example, (a) presentation of an antigen (such as in prophylactic or therapeutic vaccination), where it would be desirable to attract DCs and/or T cells to produce an increased immune response to the antigen (i.e., as an adjuvant), (b) development or direction of an immune response to a tumor, an infectious agent (e.g., virus, bacteria, parasite, fungus), diseased tissue or other antigenic agent in the subject's body, where it would be desirable to attract DCs in order to stimulate an immune response in the area affected by the tumor, infectious agent, diseased tissue, or another antigenic agent, and (c) stimulation of a change of the balance of DC and/or T cell populations at a particular site (e.g., stimulating increased infiltration of Th2 cells into the site of a Th1-mediated autoimmune condition). Thus, the chemotactic agents can be used to treat, among other conditions, bacterial, viral, fungal, and other infections, tumors and other hyperproliferative disorders, immunodeficiencies, diseases susceptible to treatment by administration of a therapeutic vaccine, and autoimmune conditions.

In other situations, such as autoimmune conditions, it can be desirable to direct or attract DCs and/or T cells away from a site where they produce an undesired effect. Thus, the agents of the invention can also be used for the treatment of conditions caused by the undesirable action of DCs and/or T cells, including autoimmune conditions.

In still other situations, it can be desirable to use the chemotactic agents of the invention to inhibit or reduce chemotaxis of DCs and/or T cells to an undesired site of action (e.g., by administering the agent in order to diminish or eliminate an endogenous in vivo chemotactic gradient, such as by administering the agent systematically).

Another embodiment of the invention is a method for activating DCs, monocytic cells, or a combination thereof, wherein the method comprises administering to the subject a pharmaceutical composition comprising a therapeutically effective amount of EDN or hPR, such that DCs, monocytic cells, or a combination thereof are activated in the subject. Activation of DCs, monocytic cells, or a combination thereof lead to the production of various mediators including cytokines, chemokines, growth factors, and soluble receptors, as described herein.

The invention also provides a method for inducing maturation of dendritic cells in a subject, wherein the method comprises administering to the subject a pharmaceutical composition comprising a therapeutically effective amount of EDN or hPR, such that maturation of dendritic cells is induced in the subject. Maturation of DCs can be evidenced by the upregulation of CD83, CD86, HLA-DR, and the functional chemokine receptor, CCR7.

Antagonists of EDN or hPR, such as polyclonal or monoclonal antibodies to EDN or hPR, soluble receptors for EDN or hPR, or fragments or derivatives of EDN or hPR, which are capable of binding to their receptors without inducing some or all of the activities induced by EDN or hPR in the absence of the antagonist can be used to counteract the effects of EDN or hPR in vivo. For example, antagonists can be administered to reduce, inhibit, or eliminate an inflammatory or autoimmune condition that is induced, in whole or in part, by EDN, hPR, or compositions thereof. Methods of producing antibodies are well known in the art. Methods of producing antagonistic fragments and derivatives also within are well known in the art and, for example, utilize known molecular biology or synthetic techniques. It is also within the ordinary skill in the art to test antagonists to determine if the antagonists compete with EDN or hPR. For example, antagonists against EDN can be tested in assays for DC chemotaxis, wherein functional antagonists can be identified by the reduction or abolition of chemotactic activity.

Accordingly, the invention also provides a method of detecting a compound that decreases an immune response in a subject (e.g., antagonists of EDN). The method comprises (a) contacting a leukocyte migration system containing EDN with the compound and (b) detecting a decrease in migration of leukocytes in the system with the compound compared to migration of leukocytes in the system without the compound. The decrease in migration of leukocytes in the system with the compound is an indication that the compound decreases an immune response in the subject. A “leukocyte migration system” refers to a microchemotaxis chamber in which chemotactic factors diluted in chemotaxis medium are placed in the wells of the lower compartment and leukocytes suspended in chemotaxis medium are added to the wells of the upper compartment. The upper and lower compartments are separated from each other by a polycarbonate membrane that traps migrating cells. The trapped cells can be stained and counted and presented as cells per high power field. A compound that decreases the migration of leukocytes toward the chamber containing a chemotactic factor decreases an immune response in a subject.

The invention also provides a method of diagnosing an inflammatory syndrome in a patient, comprising (a) obtaining a test sample from a patient; (b) determining the amount in the test sample of one or more ribonucleases; (c) obtaining a control sample from a population of healthy patients; (d) determining the amount in the control sample of the ribonucleases; and (e) comparing the amount of the ribonucleases in the test sample with the amount of ribonucleases in the control sample. An increased amount of ribonucleases in the test sample relative to the amount in the control sample indicates an inflammatory syndrome in the patient. A patient refers to a human. Inflammatory syndromes that can be diagnosed in a patient include, but are not limited to, sepsis, arthritis, allergy, enteritis, severe acute pancreatitis, emphysema, multiple organ failure, tissue or organ rejection, cardiovascular disease, infectious disease, autoimmune disease, rheumatoid arthritis, psoriasis, lupus, inflammatory bowel disease, and acute respiratory distress syndrome (ARDS).

Test samples used for performing the diagnostic method are preferably from serum, plasma, blood, lymph fluid, peripheral lymphatic tissue, or blood. Desirably the test sample contains, or has contained, leukocytes, monocytes, DCs, or Langerhans cells.

The control sample can be obtained from an organ distal to the site of local inflammation in the patient, or from a subject or subjects not experiencing or evidencing an inflammatory syndrome. An average value or range can be determined from a population of healthy individuals and used as a control value. Altered expression can be determined at any threshold that is statistically significant. For example, the threshold can be an increase relative to a control sample of 25%, 50%, or 75%.

The following examples further illustrate the invention but, of course, should not be construed as in any way limiting its scope.

### EXAMPLE 1

This example demonstrates that EDN induces chemotaxis of CD34+ cell-derived iDCs.

Natural human EDN (Newton et al., J. Natl. Cancer Inst. 90, 1787-1791 (1998)) was purified from commercial preparations of human urinary gonadotrophin as described in Sozzani et al., J. Leukoc. Biol., 66, 1-9 (1999). This purified EDN was utilized for Examples 1-7 and 12.

Dendritic cell migration was assessed using a 48-well microchemotaxis chamber as described in Yang et al., J. Immunol., 166, 4092-4098 (2001). The cells were washed three times and resuspended in chemotaxis medium (CM, RPMI1640 containing 1% BSA). Test factors (e.g., EDN) diluted with CM were placed in the wells of the lower compartment of the chamber (Neuro Probe, Cabin John, Mass.), and DCs suspended in CM at 1×10⁶ cells/mL were added into the wells of the upper compartment. The lower and upper compartments were separated by a 5 μm polycarbonate filter (Osmonics, Livermore, Calif.). After incubation at 37° C. for 1.5 hours in humidified air with 5% CO₂, the filters were removed, scraped, and stained. The cells migrated across the filter were counted with the use of a Bioquant semiautomatic counting system (which objectively determines the average cell number of six defined microscopic fields of each spot on the filter membrane) and presented as the number of cells per high power field (No./HPF).

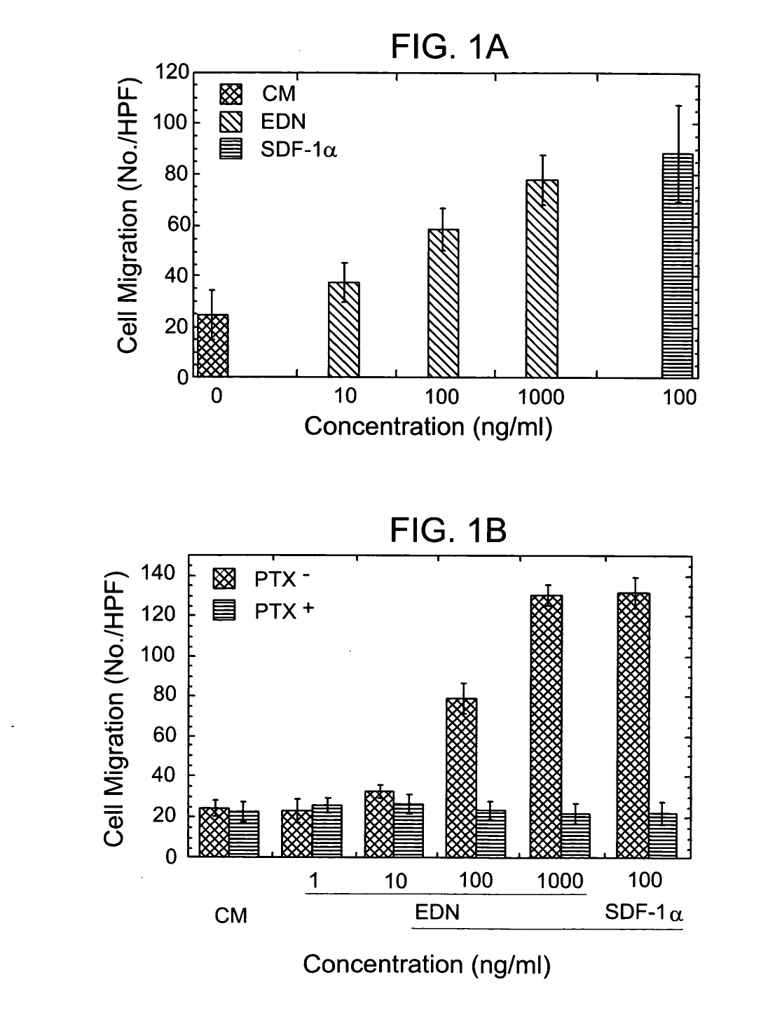

EDN was tested over a range of about 10 to about 1000 ng/mL (corresponding to about 0.6 to about 60 nM; molecular mass of EDN is about 15.5 kDa) and found to induce the migration of human CD34+ cell derived immature DCs (iDCs) in a dose-dependent manner (see FIG. 1A). The use of CM alone (e.g., no addition of EDN or another test factor) was used as a negative control. The use of a known chemoattractant, SDF-1α (100 ng/mL, PeproTech, Rocky Hill, N.J.), was used as a positive control

To address whether EDN-induced migration of iDCs was due to chemotaxis or chemokinesis, checkerboard analysis was performed. Briefly, iDCs were used at 1×10⁶ cells/mL. EDN was added to the lower wells of the chemotaxis chamber at 0, 10, 100, or 1000 ng/mL. iDCs were added to the upper wells of the chemotaxis chamber in the presence of 0, 10, 100, or 1000 ng/mL of EDN. The results are shown as the average (mean±standard deviation) of migrated DCs of triplicate wells (No./HPF) in Table 1.

TABLE 1
Checkerboard Analysis of EDN-Induced DC Migration

| EDN in the | | | | |
|---|---|---|---|---|
| Lower Wells | EDN in the Upper Wells (ng/mL) | | | |
| (ng/mL) | 0 | 10 | 100 | 1000 |
| 0 | 22 ± 3 | 24 ± 5 | 24 ± 5 | 22 ± 4 |
| 10 | 31 ± 5* | 22 ± 3 | 22 ± 5 | 22 ± 5 |
| 100 | 51 ± 5** | 35 ± 6* | 23 ± 4 | 20 ± 4 |
| 1000 | 105 ± 11** | 51 ± 7** | 36 ± 6* | 28 ± 4 |

*p < 0.005 when compared with background DC migration (22 ± 3) by Student's t test.
**p < 0.0001 when compared with background DC migration (22 ± 3) by Student's t test.

EDN, in a dose-dependent manner, induced the migration of human CD34+ cell-derived iDCs when added to the lower wells of the chemotaxis chamber. However, increasing the concentrations of EDN added simultaneously to the cells to the upper walls of the chamber not only was unable to induce directional iDC migration, but also in a dose-dependent manner abrogated iDC migration induced by EDN in the lower wells.

Thus, EDN induces migration of iDCs based on chemotaxis rather than chemokinesis.

### EXAMPLE 2

This example demonstrates that EDN-induced iDC chemotaxis is mediated by a Giα-protein-coupled receptor (GPCR).

Pertussis toxin (PTX) (Sigma, St. Louis, Mo.) is a toxin that specifically inhibits GPCR signaling by ADP ribosylating Giα-protein. To determine whether EDN-induced chemotaxis was mediated by a GPCR, iDC were incubated at 37° C. for 30 minutes in the presence or absence of PTX (200 ng/mL) prior to the chemotaxis assay described in Example 1. The migration of DC was measured, and the results set forth in FIG. 1B.

The incubation of iDC with PTX completely inhibited the migration of iDCs in response to EDN. Pretreatment with PTX did not affect the motility of iDCs, since there was no difference in spontaneous migration (in response to CM) between iDCs pretreated with or without PTX. The inhibition was not due to the preincubation per se, since incubation of iDCs in the absence of PTX did not inhibit iDC migration in response to EDN (see FIG. 1B).

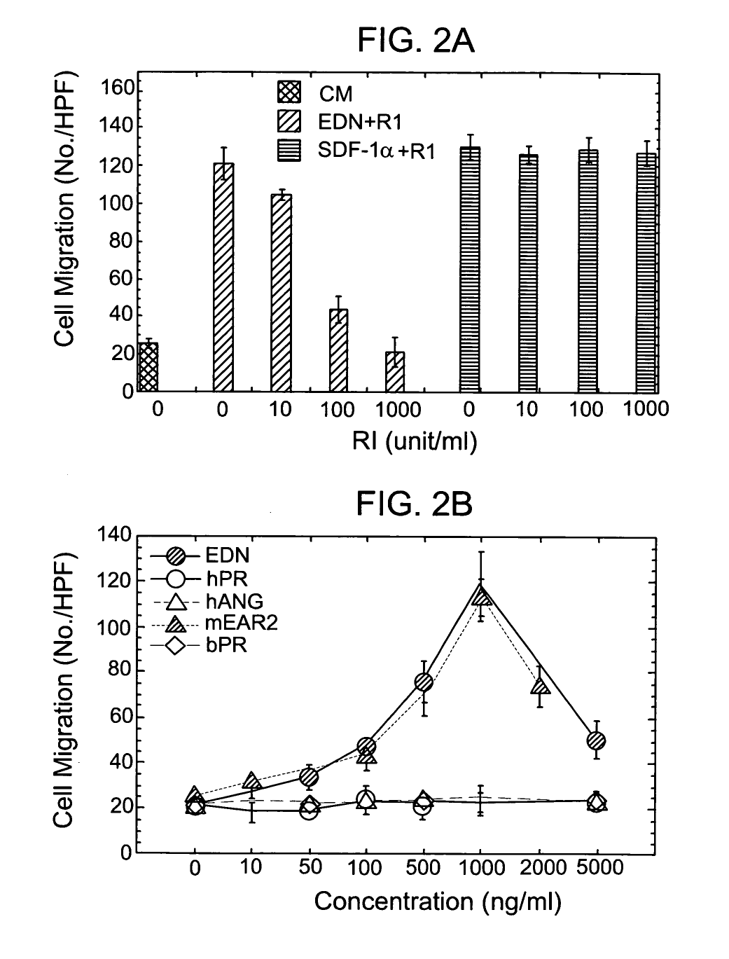

Additionally, cell migration of iDCs was evaluated by chemotaxis assay as described in Example 1, wherein EDN, mEAR2 (recombinant mEAR2 purified from culture supernatants of Spodoptera frugiperda (Sf9) insect cells infected with recombinant baculovirus encoding mEAR2), hPR, hANG (R & D System, Minneapolis, Minn.), and bPR (Sigma, St. Louis, Mo.) were added to the lower wells of the chemotaxis chamber at doses ranging from about 0 to about 5000 ng/mL. The cell migration results are set forth in FIG. 2B.

EDN, as well as mEAR2, which is a member of a cluster of ribonuclease genes that as a group are distant orthologs of EDN in mice, induced iDC chemotaxis with a bell-shaped dose-response curve typical of most GPCR-dependent chemotactic factors with an optimal dose of 1000 ng/mL (FIG. 2B), while other members of RNase family, such as hPR, hANG, and bPR had no effect on DC migration regardless of concentration (FIG. 2B).

Therefore, EDN induces chemotaxis of iDCs in a Giα-protein dependent manner.

### EXAMPLE 3

This example demonstrates that the chemotactic activity of EDN is inhibited by an RNase inhibitor.

The RNase activity and antiviral activity of EDN has previously been shown to be inhibited by an RNase inhibitor (RI). To determine whether the chemotactic activity of EDN would also be inhibited by a RI, 0, 10, 100, and 1000 units/mL of RI and 1000 ng/mL of EDN were simultaneously added to the lower wells of the chemotaxis chamber described in Example 1. The cell migration results are set forth in FIG. 2A.

The simultaneous addition of RI and EDN to the lower wells inhibited the migration of iDCs induced by 1000 ng/mL of EDN (FIG. 2A). The inhibition was specific for EDN since identical concentrations of RI did not inhibit the migration of iDCs induced by 100 ng/mL of SDF-1α (FIG. 2A). This indicates that the chemotactic activity of the EDN sample was actually due to the ribonuclease, EDN, and not a contaminant.

Therefore, the chemotactic activity of EDN is inhibited by a RNase inhibitor.

### EXAMPLE 4

This example demonstrates that EDN is selectively chemotactic for DCs.

To identify the spectrum of target cells for EDN's chemotactic activity, the changes in chemotaxis response to EDN in the course of DC differentiation and maturation were examined. Specifically, the migration of CD34+ cell-derived DC precursors (preDCs), iDCs, and mDCs and various types of leukocytes in response to EDN (0 to about 5000 ng/mL) was evaluated by chemotaxis assay as generally described in Example 1. The cell migration results are set forth in FIGS. 3A and 3B.

Precursors of DCs (preDCs) amplified from CD34+ HPC failed to respond chemotactically to EDN (FIG. 3A). This failure was not due to the lack of capacity of the preDCs to migrate, since preDCs migrated toward SDF-1α (100 ng/mL) in the same experiment (FIG. 3A). As preDCs differentiated into iDCs, they developed the capacity to migrate in response to EDN (FIG. 3A). Furthermore, mDCs still maintained the capacity to migrate chemotactically to EDN (FIG. 3A).

Additionally, whether EDN was chemotactic for DCs generated from human peripheral blood monocytes was investigated. Both monocyte-derived iDCs (Mo-iDCs) and mDCs (Mo-mDCs) migrated chemotactically in response to EDN with optimal doses at 1000 mg/mL (FIG. 3B). EDN failed to chemoattract monocytes (Mo) from which the DCs were generated, although those monocytes were fully capable of migrating to a known chemoattractant, N-formyl-methionyl-leucyl-phenylalanine (fMLP) (Figure B). Screening of human peripheral blood leukocytes revealed that EDN over a large concentration range (about 10 to about 5000 ng/mL) did not induce the migration of purified neutrophils, eosinophils, or T cells.

Thus, EDN is selectively chemotactic for human iDCs and mDCs, but not chemotactic for any other leukocytes examined.

### EXAMPLE 5

This example demonstrates that EDN and mEAR2 are chemotactic for murine DCs.

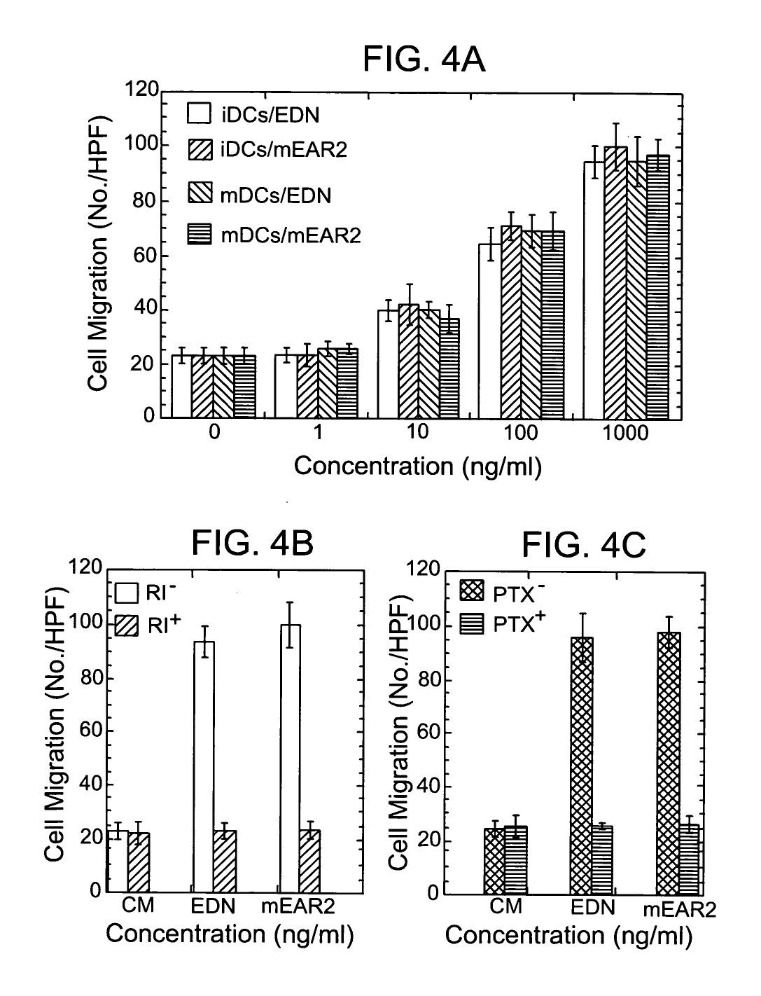

EDN and recombinant mEAR2 were produced as described in Examples 1 and 2. The migration of iDCs and mDCs generated from mouse bone marrow hematopoietic progenitor cells (HPCs) in response to EDN or mEAR2 was investigated by chemotaxis assay as described generally in Example 1, and the cell migration results set forth in FIGS. 4A, 4B, and 4C.

Both EDN and mEAR2 induced chemotaxis of iDCs and mDCs generated from mouse bone marrow HPCs (FIG. 4A). As observed with human DCs, the migration of mouse DCs in response to EDN or mEAR2 was inhibited by the presence of placental RI (FIG. 4B), or if DCs were pretreated with 200 ng/mL of PTX at 37° C. for 30 minutes (FIG. 4C).

Collectively, EDN and mEAR2 are therefore chemoattractants for both human and mouse DCs.

### EXAMPLE 6

This example demonstrates the sequence analysis of the amino acid sequences of EDN and mEAR2.

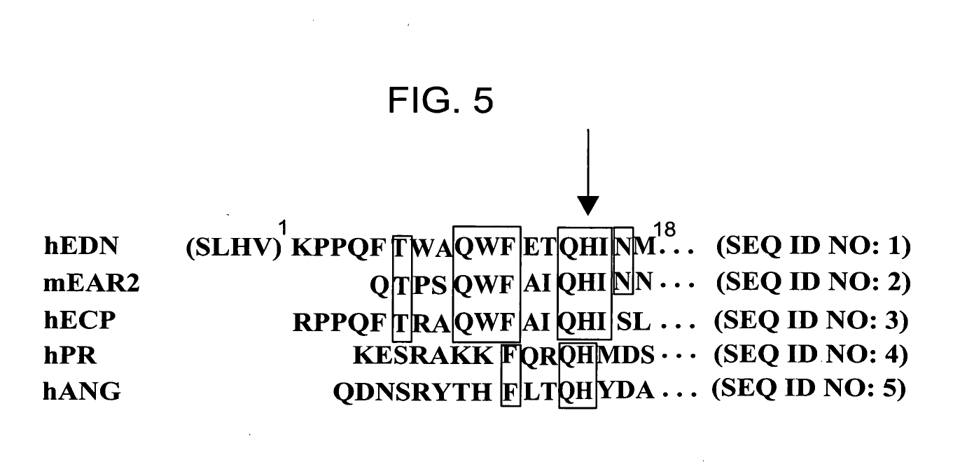

With the observation that human EDN and mEAR2 are chemotactic for DCs, but hANG and hPR are not (see Example 2 and FIG. 2B), a comparison of their sequences permitted the analysis of the region(s) of sequence most likely to play a significant role in chemotaxis of DCs. The results of such an analysis, which are set forth in FIG. 5, pointed to the amino terminal sequences of EDN and mEAR2 as having significant sequence homology to each other (8/18 residues conserved), while there was very limited homology to either hPR (3/18 residues conserved) or HANG (also 3/18 residues conserved, p<0.02 by χ2 test).

If the amino terminal sequences of EDN and mEAR2 are responsible for chemotaxis of DCs by EDN and mEAR2, human eosinophil cationic protein (ECP) should also function as a chemoattractant for DCs, given the sequence identity with both EDN and mEAR2, specifically at the amino terminus (7/18 and 9/18 residues conserved when compared to EDN and mEAR2, respectively). ECP and EDN both belong to the eosinophil-associated ribonuclease (EAR) subfamily of the RNase superfamily, and ECP, like EDN, has in vitro antiviral activity. Accordingly, it is possible that ECP, like EDN, functions as an immunoadjuvant, and would be useful in the compositions and methods of the invention.

EDN exists in two forms in vivo due to alternative splicing. The −4 form of the EDN has 4 additional amino acid residues (SLHV) on the N-terminus (FIG. 5). Both EDN with and without the N-terminal 4 additional residues can be chemotactic since both forms have essentially identical tertiary structures. Moreover, mEAR2, which lacks the corresponding N-terminal 8 residues of the −4 form of EDN (FIG. 5), is nevertheless similarly chemotactic as the −4 form of EDN. Sequence comparison of EDN and mEAR2 (both chemotactic for DCs), as well as hANG and hPR (not chemotactic for DCs), indicates that the N-terminal region (¹Q-⁹I) of mEAR2 is important for its DC chemotactic activity (FIG. 5). The ¹Q-⁹I region of mEAR2 corresponds to the ⁵F-¹³T region of EDN, indicating that the 4 additional N-terminal amino acid residues of the −4 form of EDN are not critical for its chemotactic effect.

### EXAMPLE 7

This example sets forth a procedure to demonstrate the in vivo inflammatory effects of EDN in mice.

BALB/C mice are injected subcutaneously with 1 μg of EDN and after about 4 or about 24 hours, the injection site is excised, and the extent and types of cells infiltrating the site are examined histologically. Within several hours (about 4 hours) of injection of EDN, infiltration by polymorphonuclear neutrophilic (PMN) cells and mononuclear cells in the dermis and subcutaneous tissues is seen. This result is in contrast to a single 1 μg injection of recombinant human (rh) IL-8, which produces a marked infiltration of PNM cells in the same period with little mononuclear cell infiltration. By 24 hours after injection, an even greater infiltration of PMNs and mononuclear cells is elicited by EDN. EDN is unique in attracting a considerable number of mast cells along with neutrophils to the injection site by 24 hours post-injection. Thus, EDN is capable of inducting considerable local neutrophil and mononuclear cell infiltration.

Immunohistochemical studies of sites of EDN injection in chimeric human peripheral blood lymphocyte severe combined immunodeficiency (huPBL SCID) mice are performed to establish whether there are dendritic cells in the infiltrate. A single injection of 1 μg of EDN results in the infiltration by human dendritic cells within 4 hours of injection in the mice examined. In contrast, the sites of PBS injection in control mice do not contain any human CD3+ T cells. These in vivo results support the in vitro evidence that EDN is a dendritic cell chemoattractant, and therefore, can enhance an immune response in a subject.

### EXAMPLE 8

This example demonstrates that mEAR2 induces the accumulation of DCs in vivo.

To analyze the chemoattractant activity in vivo, the number and identity of cells accumulating in response to injection of mEAR2 into the air pouches of mice were determined. Air pouches were raised on the dorsum by subcutaneous injection of 3 mL of sterile air on days 0 and 3. On day 6, mice with a well-formed air pouch were randomized into three different groups. Each mouse was injected with 1 mL of endotoxin-free PBS alone or PBS containing 1 μg/mL of hANG or mEAR2 into the air pouches. Four hours after injection of either PBS, hANG, or mEAR2 into the air pouches, mice were sacrificed, and cells infiltrating into the air pouches were washed out, stained, and counted. To evaluate the migration of DCs (CD11c and I-A/I-E double positive (Banchereau et al., Nature, 392, 245-251 (1998))) into the air pouch, a portion of the cells were double-stained with a combination of FITC-conjugated rabbit anti-mouse I-A/I-E (Clone 2G9, PharMingen, San Diego, Calif.) and PE-conjugated hamster anti-mouse CD11c (Clone HL3, PharMingen), and analyzed by FACScan flow cytometer (Becton Dickenson, San Jose, Calif.).

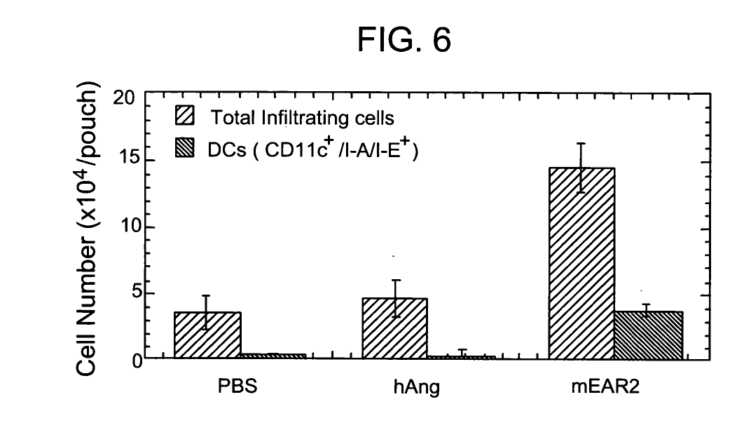

The proportion of PE/FITC-double positive cells (i.e., CD11c and I-A/I-E double positive) in the mEAR2-treated group was much higher than either the PBS- or hANG-treated group, suggesting that injection of mEAR2 into the air pouches resulted in recruitment of DCs into the air pouches. The average number of leukocytes and DCs recruited into the air pouches of each group is shown in FIG. 6. Injection of hANG did not enhance the recruitment of either total leukocytes or DCs into the air pouches. In agreement with the in vitro chemoattractant activity of EDN and mEAR2, injection of mEAR2 promoted the recruitment of DCs (FIG. 6, black bars) into the air pouches. Notably, in the mEAR-2 treated group, DCs only accounted for approximately 50% of the increase in total infiltrating cells, suggesting that leukocytes other than DCs were also recruited into the air pouches by mEAR2 injection. Staining of the cells recovered from mEAR2-injected air pouches revealed that more than 95% of the cells were of mononuclear morphology, indicating that there was no apparent granulocyte recruitment.

This analysis suggests that mEAR2 acts as a DC chemoattractant in vivo, and is indicative of EDN's in vivo effects.

### EXAMPLE 9

This example demonstrates that both EDN and hPR induce the production of various mediators by CD34+ progenitor derived iDCs in a dose- and time-dependent manner.

The genes coding for human EDN or hPR were cloned into the vector pET-11d (Novagen, Madison, Wis.) and expressed in BL21(DE3) E. coli cells (Novagen) as described in Newton et al., Antibody Engineering Lab Manual, Dubel et al., eds., 667-688 (2001). The recombinant proteins were isolated for inclusion bodies, denatured, renatured, and purified.

To characterize the capacity of EDN to induce soluble mediator production by DCs, the dose- and time-dependent effect of EDN on the production of 78 different mediators was investigated by antibody microarray-based RCA immunoassay.

Antibody microarrays were printed using a BCA-II piezoelectric microarray dispenser (Packard Biosciences, Downers Grove, Ill.) on cyanosilane-coated glass slides divided by Teflon boundaries into sixteen 0.5 cm diameter circular sub-arrays. Monoclonal antibodies for 78 analytes were dispensed in quadruplicate at a concentration of 0.5 mg/mL. Printed slides were subjected to a quality control consisting of incubation with a fluorescent-labeled anti-mouse antibody, followed by washing, scanning, and quantitation. Typically the coefficient of variability of antibody deposition in printing was <5%.

The RCA immunoassay was performed by a liquid-handling Biomek 200 robot (Beckman Instruments, Fullerton, Calif.) that was enclosed in an 80% humidified, HEPA-filtered, plexiglass chamber. For each sample, duplicates were tested either undiluted or diluted at 1:10. Twenty μL of the samples was applied to each sub-array, and an RCE immunoassay was performed as described in Schweitzer et al., Nat. Biotechnol., 20, 359-365 (2002). Slides were scanned by the use of GenePix (Axon Instruments Inc., Foster City, Calif.) at 10 μm resolution with a laser setting of 100 and PMT setting of 550. Mean pixel fluorescence was quantified using the fixed circle method in GenePix Pro 3.0 (Axon Instruments). The fluorescence intensities of 8 microarray features (duplicate sub-arrays and quadruplicate spots in each sub-array) were averaged for each feature and sample, and the resulting cytokine values as Cy5 intensity were determined. For each slide, a set of blanks was run as a negative control. Analyses were performed using a complete set of data, and the levels of all 78 analytes (cytokines, chemokines, growth factors, and soluble receptors) were analyzed as relative fluorescent intensity units of as fold increase for the purpose of comparison. The sensitivity of the RCA immunoassay was femtomolar (see Schweitzer et al., supra; Kingsmore et al., Curr. Opin. Biotechnol., 14, 74-81 (2001)): 46 (60%) of the 78 analytes had a sensitivity of ≦10 pg/mL, 24 (30.8%) had a sensitivity of ≦100 pg/mL, 5 (6.4%) had a sensitivity of ≦1 ng/mL, and 3 (3.8%) had a sensitivity of ≦10 ng/mL. Importantly, the dynamic range (approximately 3 orders of magnitude) and precision of the RCA immunoassay were similar to unamplified immunoassay (see Schweitzer et al., supra; Kingsmore et al., supra). Femtomolar sensitivity and 3 log dynamic range appears to be adequate for the measurement of most biologically relevant changes in cytokine secretion.

In the antibody microarray-based RCA immunoassay, hPR was intended to be used as a negative control, since both the chemotactic and antiviral activities of EDN were not shared by hPR. In the case of CD34+ progenitor-derived iDCs, EDN induced the production of 18 mediators (with fold increase ≧3), and hPR also induced the production of a similar set of mediators. Those mediators with a fold increase of greater than or equal to 3 are shown in Table 2.

TABLE 2
Comparison of Fold Increase in Mediator Production by
CD34⁺ Cell-Derived iDCs in Response to EDN or hPR.

| Mediators | EDN | hPR |
|---|---|---|
| ENA-78 | 91 | 26 |
| Eot2 | 1 | 1 |
| I-309 | 4 | 3 |
| IFNα | 1 | 1 |
| IL-10 | 1 | 2 |
| IL-12p40 | 32 | 4 |
| IL-12p70 | 7 | 2 |
| IL-13 | 1 | 2 |
| IL-16 | 1 | 1 |
| IL-6 | 182 | 74 |
| IP-10 | 74 | 10 |
| MCP-1 | 4 | 4 |
| MCP-2 | 26 | 8 |
| MCP-3 | 97 | 18 |
| M-CSF | 5 | 2 |
| MIG | 4 | 2 |
| MIP-1α | 14 | 7 |
| MIP-1β | 2 | 2 |
| MPIF-1 | 3 | 2 |
| NAP-2 | 4 | 2 |
| Rantes | 22 | 12 |
| sCD23 | 2 | 3 |
| TNF-α | 25 | 5 |
| sTNF-RI | 3 | 2 |

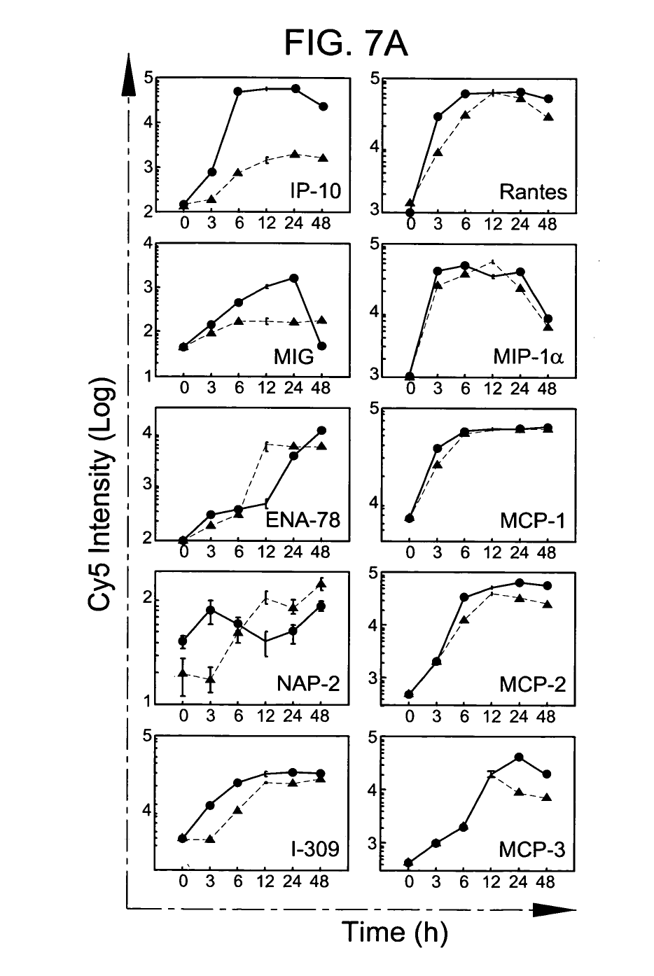

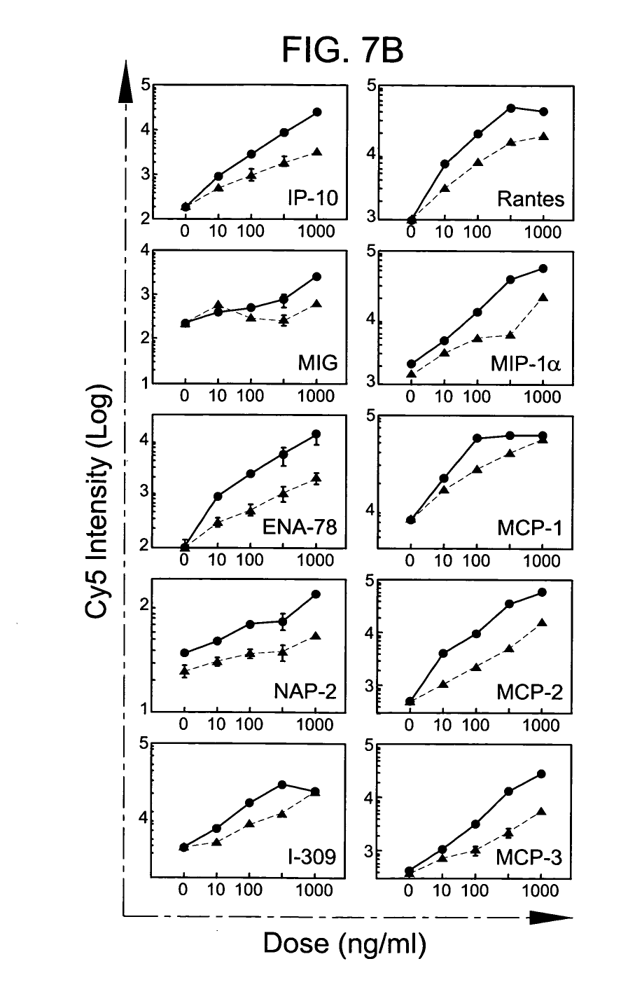

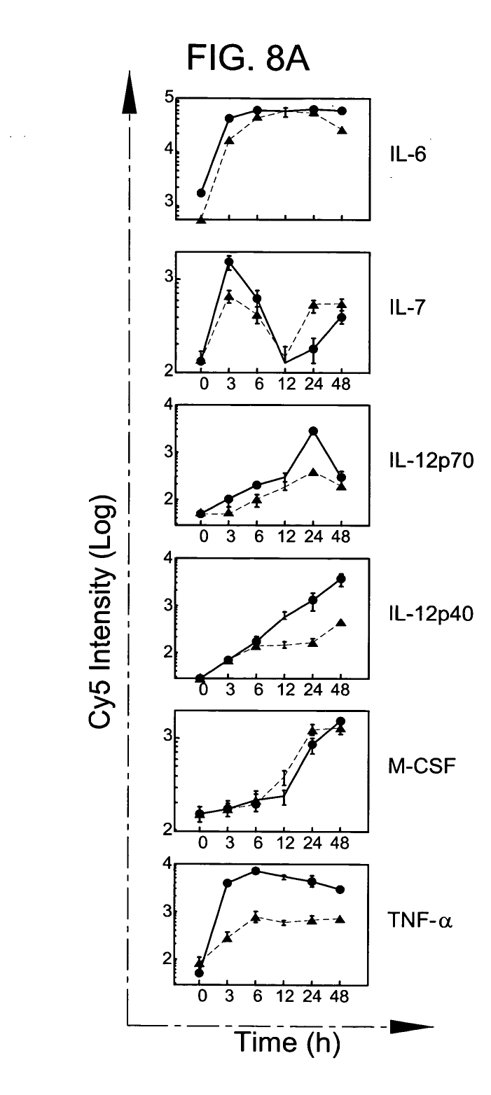

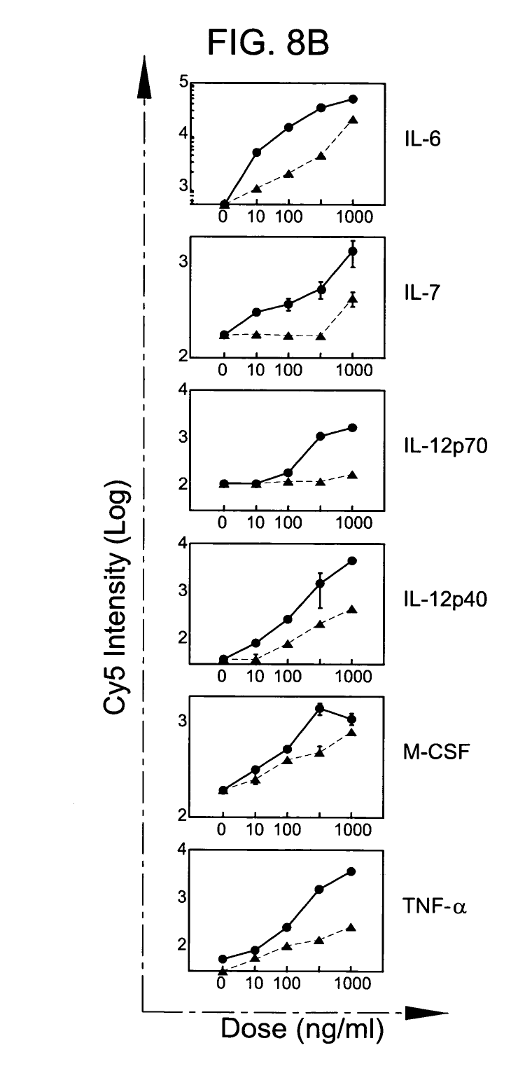

To further characterize the capacity of EDN and hPR to induce soluble mediator production by DCs, the dose- and time-dependent effects of EDN and hPR on CD34+ progenitor derived DCs were analyzed by RCA immunoassay as described in Example 9. The time-course and dose-response of EDN- and hPR-induced production of 10 selected chemokines and 5 selected cytokines by CD34+ progenitor-derived DCs are shown in FIGS. 7 and 8, respectively.

The EDN- and hPR-induced production of mediators was dose-dependent and peaked at different time points for different mediators. For example, EDN induced (a) MIP-1α, Rantes, IL-6, IL-7, and TNF-α maximally at about 3-6 hours, (b) ENA-78, IP-10, MCP-1, and I-309 at about 12 hours, (c) MIG, MCP-2, MCP-3, and IL-12p70 at about 24 hours, and (d) neutrophil activating protein (NAP)-2, IL-12p40, and M-CSF at about 48 hours. Although the induction of most cytokines and chemokines by EDN and hPR was similar or identical (e.g., MCP-1), hPR, unlike EDN, did not induce the production of MIG and IL-12p70.

Thus, both EDN and hPR induce the production of various mediators by CD34+ progenitor derived iDCs in a dose- and time-dependent manner.

### EXAMPLE 10

This example demonstrates that EDN and hPR induce the proliferation of distinct sets of mediators by monocyte-derived DCs, human monocytes, and THP-1 cells.

The antibody microarray-based RCA immunoassay was performed as described in Example 9, except monocyte-derived iDCs, human monocytes, and THP-1 cells were tested. When monocyte-derived iDCs were tested, a similar group of mediators was induced in response to EDN or hPR in a time- and dose-dependent manner. Those mediators with a fold increase of greater than or equal to 3 are shown in Table 3.

TABLE 3
Comparison of Fold Increase in Mediator Production
by Monocyte-Derived iDCs in Response to EDN or hPR.

| Mediators | EDN | hPR |
|---|---|---|
| ENA-78 | 2 | 1 |
| Eot2 | 3 | 3 |
| I-309 | 4 | 1 |
| IFNα | 5 | 0 |
| IL-10 | 6 | 0 |
| IL-12p40 | 3 | 1 |
| IL-12p70 | 2 | 1 |
| IL-13 | 3 | 1 |
| IL-16 | 0 | 3 |
| IL-6 | 181 | 15 |
| IP-10 | 67 | 6 |
| MCP-1 | 2 | 1 |
| MCP-2 | 18 | 1 |
| MCP-3 | 1 | 1 |
| M-CSF | 1 | 1 |
| MIG | 6 | 1 |
| MIP-1α | 10 | 2 |
| MIP-1β | 61 | 19 |
| MPIF-1 | 6 | 6 |
| NAP-2 | 2 | 1 |
| Rantes | 44 | 7 |
| sCD23 | 1 | 1 |
| TNF-α | 10 | 1 |
| sTNF-RI | 1 | 2 |

After 12 hours of incubation with 1 μg/mL of EDN or hPR, 16 (eotaxin (Eot)-2, I-309, interferon (IFN)α, IL-10, IL-12p40, IL-13, IL-6, IL-7, IP-10, MCP-2, MIG, MIP-1α, MIP-1β, myeloid progenitor inhibitory factor-1 (MPIF)-1, Rantes, and TNF-α) were induced by EDN, while 7 (Eot-2, IL-16, IL-6, MIP-1β, MPIF-1, Rantes, and IP-10) of the 78 mediators tested were induced by hPR (with fold increase ≧3).

EDN- and hPR-induced mediator production by monocyte-derived mDCs showed a similar dose-dependence and only slightly different kinetics. For example, IL-6, MIP-1α, MCP-1, MCP-2, Eot-2, IFNα, and TNF-α peaked at about 3-6 hours, MIP-1β, Rantes, IL-10, IL12p40, and MPIF-1 peaked at about 12 hours, and IL-13, MIG, J-309, IP-10, and IL-7 peaked at about 24-48 hours.

EDN and hPR also stimulated the production of various mediators by human peripheral blood monocytes and the monocytic cell line, THP-1. After culturing THP-1 cells from 48 hours with EDN or hPR, the production of the following mediators was observed: EDN induced the production of 28 mediators (with fold induction of ≧3), including BLC, I-309, IFNα, IFNγ, IL-10, IL-12p40, IL-13, IL-18, IL-1β, IL-1ra, soluble (s) IL-2Rα, IL-3, IL-6, sIL-6R, IL-8, IP-10, MCP-1, MCP-2, MCP-3, MDC, MIP-1α, MIP-1β, NAP-2, oncostatin (OSM), TARC, TNF-α, sTNF-R1, and urokinase-type plasminogen activator receptor (uPAR). In contrast, hPR induced the production of only 13 mediators including glial derived neurotrophic factor (GDNF), IFNα, IL-10, IL-18, IL-1β, IL-6, IL-8, IP-10, MCP-2, MDC, and MIP-1β. Although both EDN and hPR stimulated production of similar amounts of IFNα by THP-1 cells, EDN, but not hPR, induced the production of large amounts of BLC, I-309, IL-12p40, MCP-2, MIP-1α, NAP-2, OSM, TARC, TNF-α, and uPAR, which highlights the differences between EDN and hPR.

### EXAMPLE 11

This example demonstrates that the capacity of EDN and hPR to induce mediator production by iDC was not due to endotoxin contamination.

Since E. coli derived EDN and hPR samples were utilized in most of the microarray experiments, it was determined whether the observed effect was due to a possible endotoxin contamination. The endotoxin levels in the samples were measured by the use of Limulus Amebocyte Lysate Pyrogent test kit (Biowhitteker, Walkersville, Md.) following the manufacturer's protocol. The endotoxin levels of the EDN and hPR samples used in the microarray experiments were below the detection limit of the Limulus Amebocyte Lysate assay, indicating less than 0.6 ng of endotoxin per mg of protein. The highest concentrations of EDN or hPR utilized in the microarray experiments were 1 μg/mL, which should contain less than 0.6 pg/mL of endotoxin. In addition, heating samples for 30 minutes at 100° C., although having no significant effect on the IL-6-inducing capacity of liposaccharide (LPS), largely destroyed the capacity of EDN or hPR to induce IL-6 production by iDCs.

The spectra of mediator induction by EDN, hPR, and LPS from CD34+ progenitor-derived iDCs also were compared. Although EDN and hPR induced a similar battery of mediators by CD34+ progenitor-derived iDCs, LPS stimulated a different set of mediators, most of which were not induced by EDN and hPR.

Therefore, the effect of EDN or hPR on iDCs is unlikely to be due to endotoxin contamination.

### EXAMPLE 12

This example demonstrates that natural and recombinant EDNs exhibit similar cytokine-inducing effects on DCs.

Since recombinant EDN generated in E. coli is not glycosylated as in natural EDN, whether EDN purified from a natural source was also able to induce the production of cytokines and chemokines was examined. Natural human EDN was purified from commercial preparations of human urinary gonadotrophin as described in Example 1.

The antibody microarray-based RCA immunoassay was performed as described in Example 9. When human CD34+ progenitor-derived iDCs were used, both natural and recombinant EDNs induced the production of similar amounts of the ten selected cytokines.

The effects of hANG, hPR, and natural and recombinant EDNs on the production of selected cytokines (IL-6, IP-10, MCP-2, MIG, and Rantes) by monocyte-derived DCs also were compared by antibody microarray-based RCA immunoassay. Both natural and recombinant EDNs induced the production of similar amounts of the selected cytokines analyzed (IL-6, IP-10, MCP-2, MIG, and Rantes). Human ANG, although also belonging to the RNase A superfamily, did not induce the production of IL-6, IP-10, MCP-2, MIG, or Rantes as EDN and hPR did, indicating that not all members of the RNase A superfamily share the capacity to induce cytokine production by DCs.

### EXAMPLE 13

This example demonstrates the alteration of gene expression of cytokines in DCs in response to EDN or hPR.

Total RNAs were isolated from iDCs cultured in G4 medium alone or in the presence of EDN or hPR for 48 hours, reverse transcribed, and analyzed by real-time PCR using TaqMan® Cytokine Gene Expression Plate I. Of the 12 cytokines tested, EDN upregulated the mRNAs for all except IL-5 and IL-10. hPR upregulated the mRNAs for IL-1α, IL-1β, IL-4, IL-12p40, and IL-15, but unlike EDN, did not induce IL-2, IL-8, IL-12p35, IFNγ, and TNF-α. However, hPR upregulated IL-5 mRNA, which is not upregulated by EDN. Both EDN and hPR did not upregulate IL-10 mRNA. The mRNA levels of these cytokines were not in complete agreement with the protein levels, indicating that the production of some cytokines was also regulated at the translational level.

### EXAMPLE 14

This example demonstrates that EDN and hPR induce the phenotypic maturation of DCs.

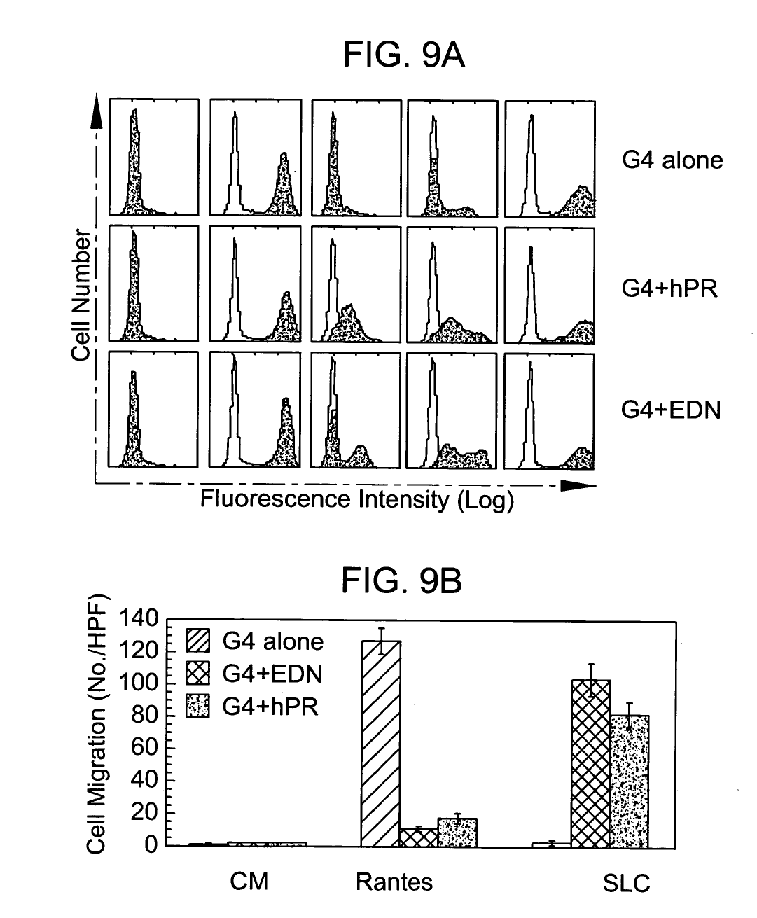

Activation of iDCs is usually accompanied by DC maturation. To determine whether treatment of iDCs with EDN or hPR could induce the phenotypic characteristics of mDCs, the following experiments were performed. Monocyte-derived iDCs were incubated in G4 medium alone in the presence of either EDN or hPR (1 μg/mL) for 48 hours at 37° C. in humidified air containing 5% CO₂ and subsequently phenotyped using flow cytometry. The results of the phenotype analysis are shown in FIG. 9A.

Immature DCs cultured in G4 alone for 48 hours maintained an immature phenotype (CD14−, CD40+, CD83−, CD86^{low}, HLA-DR^{high}) (FIG. 9A). In contrast, iDCs cultured in G4 plus hPR or EDN for 48 hours, although still CD14−, upregulated the surface expression of CD83, CD86, and HLA-DR, indicating that hPR and EDN treatment induced the maturation of DCs (FIG. 9A). In contrast to hPR, EDN-treated DCs seemed to generate two subsets of DCs differing only in the levels of surface CD83 and CD86 expression. This phenomenon was not due to donor variability since cells from three independent donors demonstrated almost identical results.

The capacity of hPR or EDN to induce DC maturation was also evidenced by the change of chemokine responsiveness of treated DCs as analyzed by chemotaxis assay as described in Example 1. The DCs cultured in G4 alone migrated in response to Rantes, but not to Secondary Lymphoid-Tissue Chemokine (SLC, PeproTech), indicating that these cells expressed functional chemokine receptors, CCR1 and/or CCR5, but not functional CCR7. However, DCs cultured in G4 in the presence of EDN or hPR lost their responsiveness to Rantes and acquired SLC responsive CCR7. Since the downregulation of a number of chemokine receptors (including CCR1 and CCR5) and upregulation of functional CCR7 is another hallmark of DC maturation, these results provide additional evidence that treatment with EDN or hPR induces the maturation of DCs.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A composition comprising (a) an immunogenic antigen and (b) eosinophil-derived neurotoxin (EDN) or human pancreatic ribonuclease (hPR).

2. The composition of claim 1, wherein the composition is a pharmaceutical composition further comprising (c) a pharmaceutically acceptable carrier.

3. The composition of claim 2, wherein the composition comprises EDN.

4. The composition of claim 2, wherein the composition comprises hPR.

5. The composition of claim 2, wherein the composition further comprises one or more cytokines, lymphokines, or hematopoietic factors.

6. A method of enhancing an immune response in a subject, comprising administering to the subject a pharmaceutical composition comprising a therapeutically effective amount of eosinophil-derived neurotoxin (EDN) or human pancreatic ribonuclease (hPR) and a pharmaceutically acceptable carrier, whereby the administration of the composition enhances the immune response in the subject.

7. The method of claim 6, wherein the subject is a mammal.

8. The method of claim 7, wherein the mammal is a human.

9. The method of claim 6, wherein the composition comprises a therapeutically effective amount of EDN.

10. The method of claim 6, wherein the composition comprises a therapeutically effective amount of hPR.

11. The method of claim 6, wherein the composition further comprises one or more or more cytokines, lymphokines, or hematopoietic factors.

12. The method of claim 6, wherein the method further comprises administering an immunogenic antigen to the subject.

13. A method of enhancing an immune response to a vaccine in a subject, comprising administering to the subject (a) a pharmaceutical composition comprising a therapeutically effective amount of eosinophil-derived neurotoxin (EDN) or human pancreatic ribonuclease (hPR) and a pharmaceutically acceptable carrier, and (b) the vaccine, whereby the composition enhances the immune response to the vaccine in the subject as compared to the immune response in the subject that results from administration of the vaccine without the composition.

14. The method of claim 13, wherein the subject is a mammal.

15. The method of claim 14, wherein the mammal is a human.

16. The method of claim 13, wherein the composition comprises a therapeutically effective amount of EDN.

17. The method of claim 13, wherein the composition comprises a therapeutically effective amount of hPR.

18. The method of claim 13, wherein the composition further comprises one or more cytokines, lymphokines, or hematopoietic factors

19. The method of claim 13, wherein the vaccine and composition are administered simultaneously.

20. A method for inducing dendritic cell (DC) chemotaxis in a subject which would benefit from such induction, wherein the method comprises administering to the subject a pharmaceutical composition comprising a therapeutically effective amount of eosinophil-derived neurotoxin (EDN) and a pharmaceutically acceptable carrier, such that DC chemotaxis is induced in the subject.

21. The method of claim 20, wherein the subject is a mammal.

22. The method of claim 21, wherein the mammal is a human.

23. A method for activating dendritic cells, monocytic cells, or a combination thereof in a subject, wherein the method comprises administering to the subject a pharmaceutical composition comprising a therapeutically effective amount of eosinophil-derived neurotoxin (EDN) or human pancreatic ribonuclease (hPR) and a pharmaceutically acceptable carrier, such that dendritic cells, monocytic cells, or a combination thereof are activated in the subject.

24. The method of claim 23, wherein the subject is a mammal.

25. The method of claim 24, wherein the mammal is a human.

26. The method of claim 23, wherein the composition comprises a therapeutically effective amount of EDN.

27. The method of claim 23, wherein the composition comprises a therapeutically effective amount of hPR.

28. A method for inducing maturation of dendritic cells in a subject, wherein the method comprises administering to the subject a pharmaceutical composition comprising a therapeutically effective amount of eosinophil-derived neurotoxin (EDN) or human pancreatic ribonuclease (hPR) and a pharmaceutically acceptable carrier, such that maturation of dendritic cells is induced in the subject.

29. The method of claim 28, wherein the subject is a mammal.

30. The method of claim 29, wherein the mammal is a human.

31. The method of claim 28, wherein the composition comprises a therapeutically effective amount of EDN.

32. The method of claim 28, wherein the composition comprises a therapeutically effective amount of hPR.

33. A method of diagnosing an inflammatory syndrome in a patient, comprising
(a) obtaining a test sample from a patient;
(b) determining the amount in the test sample of one or more ribonucleases;
(c) obtaining a control sample from a population of healthy patients;
(d) determining the amount in the control sample of the ribonucleases;
(e) comparing the amount of the ribonucleases in the test sample with the amount of ribonucleases in the control sample;
wherein an increased amount of ribonucleases in the test sample relative to the amount in the control sample indicates an inflammatory syndrome in the patient.


SEQUENCE LISTING (5 sequences)

SEQ ID NO: 1 - protein, 22 aa, Homo sapiens
SLHVKPPQFTWAQWFETQHINM

SEQ ID NO: 2 - protein, 14 aa, Mus musculus
QTPSQWFAIQHINN

SEQ ID NO: 3 - protein, 18 aa, Homo sapiens
RPPQFTRAQWFAIQHISL

SEQ ID NO: 4 - protein, 15 aa, Homo sapiens
KESRAKKFQRQHMDS

SEQ ID NO: 5 - protein, 16 aa, Homo sapiens
QDNSRYTHFLTQHYDA